SEQUENCE LISTING (4 sequences)

SEQ ID NO: 1 - protein, 8 aa, synthetic construct
FKGGERCG

SEQ ID NO: 2 - protein, 14 aa, synthetic construct
NQEQVSPLGGERCG

SEQ ID NO: 3 - protein, 10 aa, synthetic construct
RGDSPGERCG

SEQ ID NO: 4 - protein, 16 aa, synthetic construct
GCRDGPQGIWGQDRCG

(19) United States
(12) Patent Application Publication
(10) Pub. No.: US 2025/0134957 A1
(43) Pub. Date: May 1, 2025

(54) MODULAR BIOMATERIAL PLATFORM LOCAL, INJECTABLE DELIVERY OF A DRUG AND METHODS OF MAKING AND USING SAME

(71) Applicant: Duke University, Durham, NC (US)

(72) Inventors: Holly Lewis, Durham, NC (US); Tatiana Segura, Durham, NC (US); Linda Cendales, Durham, NC (US); April Espinoza, Durham, NC (US)

(21) Appl. No.: 18/927,395

(22) Filed: Oct. 25, 2024

Related U.S. Application Data
(60) Provisional application No. 63/546,082, filed on Oct. 27, 2023.
(60) Provisional application No. 63/546,961, filed on Nov. 2, 2023.

(51) Int. Cl. A61K 38/17; A61K 9/16; A61K 38/36; A61K 38/37; A61K 39/395; A61P 37/06

(52) U.S. Cl. CPC A61K 38/1774; A61K 9/1641; A61K 9/1658; A61K 38/36; A61K 38/37; A61K 39/3955; A61P 37/06

(57) ABSTRACT

Microgel assemblies provide an injectable and tunable microporous scaffold for immune cell modulation. Leukocytes from graft recipients can migrate into and integrate within the microgel assembly to induce immunological tolerance for the graft without the need for chronic immunosuppressive therapy. The microgel assembly can be used to treat for tissue graft and to reduce an alloreactive immune response following a tissue graft.

### CROSS-REFERENCE TO RELATED APPLICATIONS

This application claims the benefit of U.S. Provisional Application No. 63/546,082, filed on Oct. 27, 2023, and U.S. Provisional Application No. 63/546,961, filed Nov. 2, 2023, both of which are incorporated by reference in their entirety.

### STATEMENT OF FEDERAL SUPPORT

This invention was made with government support under Grant no. 1R38AI140297-01 awarded by the National Institute of Allergy and Infectious Disease within the Department of Health and Human Services. The government has certain rights in the invention.

### SEQUENCE LISTING

This application contains references to amino acid sequences and/or nucleic acid sequences which have been submitted concurrently herewith as the sequence listing XML file entitled “822506 us_SequenceListing.xml”, file size 4,500 bytes, created on Oct. 22, 2024. The aforementioned sequence listing is hereby incorporated by reference in its entirety pursuant to 37 C.F.R. § 1.52 (e) (5).

### FIELD OF INVENTION

The present disclosure generally relates to microporous microgel assemblies for modulating an immune response and tissue engraftment.

### BACKGROUND

Vascularized composite allotransplantation (VCA) restores skin, muscle, bone and nerve defects that cannot be rebuilt with autologous tissue. VCA has emerged as the most appropriate means of restoring form and function for select individuals following catastrophic injury or limb loss. VCA is the allogeneic transplant of peripheral tissue including skin, muscle, nerve, bone, as a functional unit (e.g. hand). In general, many of the technical aspects of VCA have been resolved, following principles derived from autologous limb replantation and free flap reconstructive surgery. However, unlike autologous grafts, all VCA grafts are rejected through allospecific T cell dependent processes known as rejection unless the recipient immune response is impaired in some way, and the optimal immune management of VCA recipients remains undefined. Immune management of VCA to prevent rejection is the most pressing barrier to VCA implementation. Despite the benefits of VCA, patients are burdened by lifelong immunosuppression, with risks including hyperglycemia, neuropathy, infections, and cancer. Multi-drug regimens include broadly-acting calcineurin inhibitors (CNI) like tacrolimus, antimetabolites such as mycophenolate and glucocorticosteroids. About 85% of VCA recipients experience rejection within the first year, a rate greatly exceeding all other transplants. Furthermore, essentially all VCA recipients experience immunosuppressive side effects, most of which are related to the use of calcineurin inhibitors (tacrolimus) and steroids. Despite best efforts, no immunotherapy completely prevents chronic graft loss, due to infiltration of alloreactive T cells and antibody-mediated rejection. All VCAs experience allospecific T cell-mediated rejection. Improving immunotherapy in VCA is the most pressing barrier to its clinical implementation.

Granular hydrogels are hydrogels composed of densely packed microgels (microgel assemblies). The microgels in turn are micron sized hydrogels that can range from 20 to 500 μm in size and can be of any shape including irregular shaped and spherical shape. Granular hydrogels can be further stabilized through interlinks between individual microgels to form a more mechanically stable structure. Due to the size of the microgels (20 to 500 μm in diameter) an interconnected micron-sized void network is established, allowing for immune cell infiltration and modulation in wounds of skin and brain. In skin, granular hydrogels augment repair via regeneration of blood vessels and lymphatics. induced by granular hydrogels involves recruitment of CD11b⁺ monocytes, but these effects are lost in mice without T cells suggesting the granular hydrogels coordinate both innate and adaptive immunity. Subcutaneous and wound-treatment with granular hydrogel modifies the polarization of macrophages, including the expression of costimulation and MHC molecules, which is of key relevance for transplantation.

Packed microgels form a microporous scaffold structure after application via a syringe or pipette in situ within the body to enable injectable therapies for immune cell modulation over sustained periods. Interlinking of the microgels is not necessary to form this structure because the tissue constrains the microgels locally. Due to the size of the microporous scaffold structure (e.g., 20 to 500 μm in diameter), an interconnected micron-sized void network is established, allowing for immune cell infiltration and modulation in wounds of skin and damaged tissue. Such materials can circumvent the need for material degradation before tissue ingrowth by providing a stably linked interconnected network of micropores for cell migration and bulk integration with surrounding tissue. Packed microgels can be designed to include lattices of microparticle polymer building blocks that may then be annealed to one another via surface functionalities to form an interconnected microporous scaffold. Microporous scaffold structure can be injected and molded to any shape to provide a mechanically stable scaffold of interconnected micropores for cell migration and bulk integration with surrounding tissue. The polymer network within the microgels can be crosslinked with peptides that can be L or D chirality. An example of microgel assemblies can be found in U.S. Pat. No. 10,849,988, which is incorporated herein by reference.

Microgel assemblies may have significant impact in other solid organ transplants (SOT). Due to mutual reliance on immunosuppression, novel therapies for VCA tolerance-induction are relevant to other organ transplants (kidney, liver, heart). Innovative approaches are needed to design therapeutics that act locally at the organ interface, limit toxicities and induce central tolerance. Recent advances in tissue engineering have shown biomaterials can play an integral role in hindlimb and islet cell transplantation by coordinating immune signals and drug delivery. The method described herein builds on work using polymeric hydrogel scaffolds. The microgel assemblies described herein incorporate immune modifying drugs currently used in clinical transplantation but delivered locally. The disclosed methods redeploy an effective wound-healing biomaterial as a transplant immunotherapy in a next-generation approach toward in vivo immune-engineering.

### SUMMARY OF THE INVENTION

Disclosed herein are microgel assemblies comprising a plurality of microgels, which comprise microgel particles, peptides, and one or more immune modifying agent. The microgel may comprise peptides having only D-amino acids or L-amino acids. The microgel assemblies may comprise only microgels with D-amino acids, only microgels with L-amino acids, or a mixture of microgels with D-amino acids and beads with L-amino acids. The microgel assemblies may further comprise thrombin, Factor XIII, and/or an additional immunosuppressive agent.

Also disclosed herein is a method of engrafting tissue in a subject comprising providing tissue to a location on or in the subject and providing the microgel assemblies described herein to the location of the graft. The tissue may be a single tissue type (e.g., skin or muscle graft) or a combination of tissues (e.g., VCA, such as limb reattachment or replacement).

Also disclosed herein is a method for reducing an alloreactive immune response to engrafted donor tissue comprising providing donor tissue to a location on or in the subject and providing the microgel assemblies described herein.

### BRIEF DESCRIPTION OF THE DRAWINGS

FIG. 1 depicts an exemplary embodiment of the methods described herein.

FIG. 2 depicts a skin graft with microgel assemblies composed of L and D chirality peptides (referred to as L/D-MAP) (solid arrows) and accompanying hair follicle neogenesis (hatched arrows).

FIGS. 3A-3C show the number of hair follicles per section (FIG. 3A), the number of sebaceous glands per section (FIG. 3B), and the % failure force (FIG. 3C) during wound healing with L-microgel assemblies (L), D-microgel assemblies (D), or 1:1 L/D microgel assemblies (R).

FIGS. 4A-4B show the number of live CD11b⁺ cells in the microgel assemblies composed of L or D-chirality peptide crosslinkers following implantation (FIG. 4A) and the percentage of F4/80⁺ cells of that population (FIG. 4B) on days 4, 7, and 14 post implantation.

FIG. 5 depicts the percentage of cells within the injected microgel assemblies composed of L- or D-chirality peptide crosslinkers that are macrophages.

FIGS. 6A-6B depict the mean fluorescence intensity (MFI) of CD11b⁺/F4/80⁺ cells that implanted in microgel assemblies composed of L or D-chirality peptide crosslinkers that co-express CD86 (FIG. 6A) or Arg1 (FIG. 6B).

FIGS. 7A-7B depict macrophages grown on microgel assemblies described herein (FIG. 7A) and a scatterplot showing F4/80 and CD11b expression (FIG. 7B) of the cells implanted within the microgel assemblies.

FIG. 8 depicts the population distribution of leukocytes that populate microgel assemblies composed of L- or D-chirality peptide crosslinkers following subcutaneous implantation in a mouse.

FIGS. 9A-9E show allogenic skin graft with microgel assemblies applied to the recipient prior to transplantation.

FIGS. 10A-10D depict the percent of total recipient T_{eff} (FIG. 10A), recipient T_{reg} cells (FIG. 10B), recipient dendritic cells (FIG. 10C) in the axillary draining lymph node and the number of recipient monocytes in inguinal draining lymph node (FIG. 10D) following MHC-matched skin graft treated with microgel assemblies composed of equal fraction of microgels composed of L and D-chirality peptide crosslinkers or vehicle control.

FIGS. 11A-11B shows the storage modulus (G′) (FIG. 11A) and loss modulus (G″) (FIG. 11B) of microgel assemblies interlinked with factor XIIIa transglutaminase and human, macaque, and murine tissue.

FIG. 12 depicts recipient and donor skin following MHC-matched skin transplantation on post operative days 4 and 7 in mice receiving microgel assemblies composed of L-chirality peptides (LMAP), D-chirality peptides (DMAP), or a microgel assembly with a 1:1 mixture of microgels comprising L-chirality peptides and microgels comprising D-chirality peptides (RMAP).

FIGS. 13A-13C show the percent engraftment (FIG. 13A) and fold change in epidermal (FIG. 13B) and dermal (FIG. 13C) thicknesses following MHC-matched skin graft treated with microgel assemblies composed of L- or D-chirality peptide crosslinkers, or an equal fraction of 1:1 L/D-microgels (labeled R).

FIGS. 14A-14D depict the number of CD3e⁺ T cell donor specific antigens (FIGS. 14A and 14B) and CD45R⁺/B220⁺ B cell donor specific antigens (FIGS. 14C and 14D) following MHC-mismatched or MHC-matched skin graft treated with microgel assemblies composed of 1:1 L/D-microgels (labeled R) or vehicle control.

### DETAILED DESCRIPTION

In the description of the illustrative embodiments, reference may be made to the accompanying drawings which form a part hereof, and in which is shown by way of illustration or a specific embodiment in which the invention may be practiced. It is to be understood that other embodiments may be utilized, and structural changes may be made without departing from the scope of the present invention.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. All publications, patents, and patent applications cited herein are hereby incorporated by reference in their entirety for all purposes.

### Microgel Assemblies

Disclosed herein are microgel assemblies comprising a plurality of microgels, wherein each microgel includes a polymeric network crosslinked through peptides and chemical groups that interlink the microgel particles to each other. In some embodiments, the microgel assemblies further include one or more immune modifying agents. In some embodiments, the crosslinked polymer within the microgel may be polyethylene glycol (PEG) or hyaluronic acid (HA). In further embodiments, the microgel assemblies may include an additional agent, such as thrombin, Factor XIII, or both. The peptides forming the polymeric network within each microgel of the microgel assemblies may be chiral. In some embodiments, the peptides within each bead may include only D-amino acids or only L-amino acids. Microgels assemblies that contain peptides with only D-amino acids may also be referred to as D-microgels or D-MAP. Likewise, microgel assemblies that contain peptides with only L-amino acids may also be referred to as L-microgels or L-MAP. Microgel assemblies that contain a mixture of D-microgels and L-microgels may also be referred to as L/D-microgels, L/D-MAP, racemic microgels, racemic-MAP, or R-MAP.

In some embodiments, the polymer may be a PEG polymer. The PEG polymer may be available as 2, 4, or 8 arm molecules and may provide a maximum of 8 sites for modification and/or crosslinking per molecule. In alternate embodiments, the polymer may be HA polymer. The HA polymer may be modified to contain about 77 acrylate groups per molecule (HA-ACR). Accordingly, HA may provide at least 9 times more sites for modification and/or crosslinking than PEG. Having more sites available for modification/crosslinking may result in a wider range of pharmaceutically active agents (e.g., immune modifying agents) without compromising crosslinking density. For example, with even 77 acrylates per chain (48% modification of the—COOH groups in HA), HA hydrogels may be completely degradable by hyaluronidase. In some embodiments, PEG-vinyl sulfone (PEG-VS) may be used. PEG is a synthetic polymer widely used in biomedical applications ranging from implant coatings to drug delivery and tissue engineering. Additionally, PEG is used as an over-the-counter and inpatient medication to mediate gastrointestinal motility (Miralax®). These wide applications (without a prescription) are a testament to the molecule's wide acceptability to both regulatory bodies and patient populations. Because PEG is biologically inert, it can serve as a blank slate to display bioactive signals and to aid in stem cell differentiation or renewal. Further, both HA and PEG polymers may be highly hydrated in water and have low protein absorption to their backbone, which is ideal for the synthesis of biomaterials with a very defined composition. The synthetic approach used to crosslink HA and PEG polymers into hydrogels to allow for the encapsulation of infiltrating leukocytes (e.g., T cells, B cells, macrophages, Langerhans cells, etc.). In some embodiments, prior to crosslinking, the HA-ACR or PEG-VS may be modified with integrin binding peptides to further enhance cellular recruitment and immune-coordination of the re-epithelializing wound.

The microgel assemblies disclosed herein may contain microgels crosslinked with peptides having a single chirality. For example, the microgel assembly may be crosslinked with only peptides having D-chirality or only peptides having L-chirality. Microgel assemblies comprising only peptides having D-chirality may also be referred to as D-microgels or D-MAP. Microgel assemblies containing only peptides having L-chirality may also be referred to as L-microgels or L-MAP. In alternate embodiments, the microgel assembly may include a mixture of L-microgels and D-microgels beads and may be referred to as L/D-microgels, L/D-MAP, or racemic microgel assemblies (e.g., R-MAP). The ratio of microgels comprising peptides with L-chirality and microgels comprising peptides with D-chirality in the L/D-MAP is not particularly limited. In exemplary embodiments, the ratio of microgels containing L-amino acids:microgels containing D-amino acids in the L/D-MAP may be about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, or about 1:10. In a particular embodiment, the ratio of the microgels containing L-amino acids to microgels containing D-amino acids in the L/D-MAP may be about 1:1 or 1:1.

The microgel particles may create an injectable scaffold in which the immune modifying agents can be pre-loaded by either the solvent associated with the hydrogel or covalently linked to the hydrogel backbone. Thus, the microgel assembly can be modified (e.g., by altering the microgel particle or particle fraction or adjusting the relative amounts of the microgel particles and the peptides) to adjust or tune the release rate of the immune modifying agent and/or any additional immunosuppressive agent. The release profiles of the immune modifying agent and/or additional immunosuppressive agent can be engineered according to the desired release rate by adjusting the microgel particles, peptides that crosslink the microgel particles, or the ratio between the two. The release profile may be rapid, slow over time, or induced by an external stimulus is applied (e.g., light such as ultraviolet light). In additional embodiments, the microgel assembly may incorporate additional chemical bonds sensitive to microenvironmental changes in oxygenation, reduction, or acidosis may be included so an additional stimulus may trigger hydrogel degradation or cargo release. Inflammatory wounds may, at times, have acidic, alkaline, or highly reducing microenvironments (e.g., due to production of reactive oxygen species, (ROS)). In some embodiments, the microgel assembly may include chemical linkages that are designed to release a drug when in the presence of ROS produced by inflammatory immune cells, which may enable not only in situ immune-engineering, but also on-demand the microgel assembly degradation and cargo released. The modifications to the microgel assembly may enable the microgel assembly to be tuned to achieve the optimal immune response and training to encourage graft acceptance, reduce the likelihood for graft rejection, and induce tolerance towards the grafted or donor tissue.

The peptides are not particularly limited and may crosslink the PEG or HA polymers to form the microgel assembly. The peptide may be K peptide: Ac-FKGGERCG-NH₂ (SEQ ID NO: 1); Q peptide: Ac-NQEQVSPLGGERCG-NH₂ (SEQ ID NO: 2); RGD peptide: Ac-RGDSPGERCG-NH₂ (SEQ ID NO: 3); and MMP: Ac-GCRDGPQGIWGQDRCG-NH₂ (SEQ ID NO: 4). Alternatively, K peptide may refer to a peptide sequence having a lysine (K). Alternatively, Q peptide may refer to a peptide sequence having a glutamine (Q). Alternatively, RGD peptide may refer to a peptide sequence having an arginine-glycine-aspartic acid (RGD) domain. All peptides within a microgel may be composed of L or D amino acids. The microgel assembly disclosed herein may be a plurality of individual microgels.

The one or more immune modifying agents may be an agent that promotes, enhances, depresses, or inhibits an immune response. The immune modifying agent may be a pro-inflammatory, an anti-inflammatory, and/or a tolerogenic agent. In some embodiments, the immune modifying agent may be an compound, protein, or nucleic acid that modulates one or more of PD-1, PD-L1, CTLA4, 4-1BB, CD40L, calcinurin, inosine-5′-monophosphate dehydrogenase (IMPDH), mammalian target of rapamycin (mTOR), janus kinase (JAK), or combinations thereof. PD-1 modulators may include, but are not limited to, anti-PD-1 antibodies (e.g., nivolumab, pembrolizumab, cemiplimab, dostarlimab, retifanlimab, toripalimab, vopratelimab, spartalizumab, camrelizumab, sintilimab, tislelizumab, INCMGA00012, AMP-224, AMP-514, acrixolimab), PD-1 inhibitors, or PD-1 agonists (e.g., roslinimab). PD-L1 modulators may include, but are not limited to, anti-PD-L1 antibodies (e.g., atezolizumab, avelumab, durvalumab, KN035, cosibelimab, AUNP12, BMS-986189), PD-L1 inhibitors, or PD-LI agonists. CTLA4 modulators may include, but are not limited to, anti-CTLA4 antibodies (e.g., ipilimumab, tremelimumab), CTLA4 inhibitors (e.g., abatacept, belatacept, CLTA4-Ig and CTLA4-Ig-like molecule), or CTLA4 agonists. 4-1BB modulators may include, but are not limited to, anti-4-1BB antibodies (e.g., utomilumab, urelumab), 4-1BB inhibitors, or 4-1BB agonists (e.g., 4-1BB ligand). CD40L modulators may include, but are not limited to, anti-CD40L antibodies, CD40L inhibitors, or CD40L agonists. Calcinurin modulators may include, but are not limited to, anti-calcineurin antibodies, calcineurin inhibitors (e.g., cyclosporin, voclosporin, pimecrolimus, tacrolimus), or calcineurin agonists. IMPDH modulators may include, but are not limited to, anti-IMPDH antibodies, IMPDH inhibitors (e.g., mycophenolate mofetil, FF-10501-01), or IMPDH agonists. mTOR modulators may include, but are not limited to anti-mTOR antibodies, mTOR inhibitors (e.g., sirolimus, temsirolimus, everolimus, ridaforolimus, rapalogs, torin-1, torin-2, vistusertib), or mTOR agonists. JAK modulators may include, but are not limited to, anti-JAK antibodies, JAK inhibitors (ruxolitinib, tofacitinib, oclacitinib, baricitinib, peficitinib, upadacitinib, fedratinib, delgocitinib, filgotinib, abrocitinib, pacritinib, deucravactinib, ritlecitinib, momclotinib, brepocitinbib, cerdulatinib, decernotinib, izencitinib, gandotinib, gusacitinib, lestaurtinib, povorcitinib, ropsacitinib, zasocitinib), or JAK agonists. In a specific embodiment, the immune modifying agent is a CTLA4 inhibitor, such as belatacept. In additional embodiments, the microgel assembly may further comprise an immunosuppressive agent in addition to the immune modifying agent described above. The additional immunosuppressive agent may be one described previously or another immunosuppressive agent.

In a particular embodiment, the microgel assembly may comprise belatacept or CTLA4-Ig, PEG, a 1:1 mixture of D-microgels and L-microgels (e.g., L/D-MAP), thrombin, and Factor XIII.

### Method of Engrafting Tissue

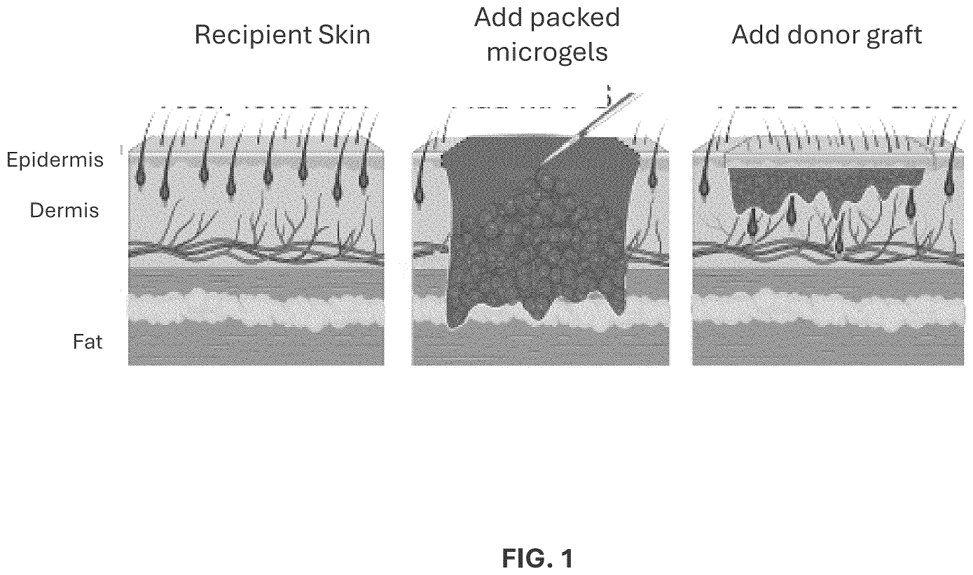

Also disclosed herein is a method for engrafting tissue in a subject including the steps of: providing tissue to a location on or in the subject, and the providing microgel assembly described above to the location. The engrafted tissue may be autologous tissue (e.g., tissue from the subject receiving the graft) or heterologous tissue (e.g., tissue from a donor other than the subject). In various embodiments, the heterologous tissue may be syngeneic to the subject receiving the graft. In other embodiments, the heterologous tissue may be allogeneic to the subject receiving the graft. In alternate embodiments, the engrafted tissue may be from a different species from the subject. An exemplary embodiment of the methods described herein is shown in FIG. 1.

As used herein, “subject” refers to an animal, such as a mammal, bird, or reptile that receives the engrafted tissue. The subject may include, but is not limited to, human, ape, monkey, companion animal (e.g., dog, cat), rodent (e.g., mouse, rat, rabbit, guinea pig, hamster, gerbil), bird, and farm animal (e.g., horse, cow, pig, sheep, goat, lamb, ox). The subject may be a domesticated animal, a non-domesticated animal, or a wild animal.

As used herein, “donor” refers to the source of the tissue grafted in or onto the subject. The donor may be the same as the subject (e.g., for graft of autologous tissue), the same species as the subject (e.g., for graft of allogeneic, heterologous tissue), or a different species (e.g., for graft of xenogenic, heterologous tissue). The donor may include, but is not limited to, human, ape, monkey, companion animal (e.g., dog, cat), rodent (e.g., mouse, rat, rabbit, guinea pig, hamster, gerbil), bird, or farm animal (e.g., horse, cow, pig, sheep, goat, lamb, ox). The donor may be a domesticated animal, a non-domesticated animal, or a wild animal. “Donor tissue” refers to tissue from the donor that is engrafted in or onto the subject.

As used herein, the term “tissue” generally refers to any biomatter provided to the subject and encompasses both a single tissue type (e.g., a plurality of cells having the same origin and/or associated with a particular organ, such as skin, muscle, liver, etc.) or multiple tissue types (e.g., a plurality of cells having different origins and/or associated with multiple organs, such as an organ system, limb, or body part). In various embodiments, the tissue to be transplanted or engrafted may include, but is not limited to, one or more of skin, kidney, bone, liver, spleen, heart, lung, cornea, muscle, ligament, tendon, fascia, nerve, lymph (including axillary and inguinal lymph nodes), blood vessels, lymph node, or combinations thereof. In some embodiments, the engrafted or transplanted tissue may include, but is not limited to, limb transplant (including forclimb, hindlimb, arm, leg, upper extremity, or lower extremity), hand transplant, foot transplant, paw transplant, hoof transplant, abdominal wall transplant, intestinal transplant, face transplant, uterus transplant, penile transplant, stomach transplant, liver transplant, kidney transplant, skin transplant, hip transplant, corneal transplant, eye transplant, heart transplant, lung transplant, or combinations thereof. In embodiments where the donor is the same as the subject, the engrafted tissue may be to replace a body part that has been partially or fully severed or otherwise damaged. In such embodiments, the method described herein may include replacing and/or reattaching severed or partially tissue, such as severed nerve, bone (including calvarial bone), tendon, ligament, scalp, skin, muscle, fat, fingers, toes, hands, fect, limbs (including forclimb, hindlimb, arm, leg, upper extremity, or lower extremity), and the like.

In various embodiments, the microgel assembly may be provided to the subject at the location of the transplant before placement of the engrafted tissue. Alternatively, the microgel assembly may be applied to said tissue prior to engraftment of the tissue and the combination of the tissue with the microgel assembly may be provided to the patient. Where necessary or appropriate, the microgel assembly may be reapplied to the transplant site after the tissue engraftment a second, third, fourth, or subsequent time to facilitate recovery from the transplant and prevent or inhibit rejection of the graft.

Also disclosed herein is a method for reducing an alloreactive immune response to engrafted tissue including the steps of: providing donor tissue to a location on or in the subject, and providing microgel assembly described above to the location. The method may include providing L-MAP, D-MAP, or L/D-MAP. Reduction of an alloreactive immune response includes, but is not limited to, reducing number of alloreactive B cells (e.g., CD45R⁺ B cells) or alloreactive T cells (e.g., CD3⁺ T cells). In other embodiments, reduction of an alloreactive response may include, but is not limited to, reduction or inhibition of production of pro-inflammatory cytokines and chemokines (e.g., interleukin (IL)-1, IL-2, IL-12, IL-18, tumor necrosis factor (TGF)-α, interferon (IFN)-γ, granulocyte-macrophage colony stimulating factor (GM-CSF), CXCL-8, CXCL-10, CCL2, CCL3, CCL4, CCL5, and CCL11). Alternatively, reduction in an alloreactive response may include enhancing production and or expression of anti-inflammatory cytokines or chemokines or regulatory cytokines or chemokines (e.g., IL-4, IL-6, IL-10, IL-11, IL-13, IL-17, IFN-α, and transforming growth factor (TGF)-β). In some embodiments, the microgel assembly, or the method of reducing an alloreactive immune response, may include induction of tolerance. The tolerance may include, but is not limited to, generation of regulatory T cells (T_{regs} or suppressor T cells), regulatory B cells (B_{regs}), or tolerogenic dendritic cells, or induction of T cell or B cell anergy.

### EXAMPLES

EXAMPLE 1: Microgel assemblies may deliver antibody-based immunotherapy in a transplant microenvironment. Injectable microgel assemblies may be synthesized in three stages. In the first stage, polyethylene glycol may be modified with vinyl sulfone groups (PEG-VS) and three peptides: a cell adhesion peptide (RGD), and two FXIII substrates: K-peptide Ac-FKGGERCG-NH₂ (SEQ ID NO: 1) and Q-peptide Ac-NQEQVSPLGGERCG-NH₂ (SEQ ID NO: 2). In the second stage, peptide-modified PEG-VS may undergo Michael addition with dicysteine-containing peptide, Ac-GCRDGPQGIWGQDRCG-NH₂ (SEQ ID NO: 4), which is protease-labile. This MMP-labile peptide can contain either L- or D-enantiomers. Formation of hydrogel gel beads may occur in an aqueous droplet surrounded by oil, generated via microfluidics. In the third stage, the microgel assemblies may be linked by FXIII to form a porous scaffold. Mechanical properties of the microgel assemblies can be modulated by altering polymer stoichiometry and degree of interlinking.

Embodiments of the microgel assemblies were tested in a mouse excision wound healing model as previously described. See Griffin, et al. Nat Mater, 2021 April; 20 (4): 560-569. Briefly 10-week old female SKH1 mice (Charles River Laboratories; n=6), or 10-week old female C57Bl/6 (B6) or B6.Rag1^{-/-} mice (Jackson Laboratories; n=4 twice) were anesthetized using continuous application of aerosolized isoflurane (1.5 vol %) throughout the duration of the procedure and disinfected with serial washes of povidone-iodine and 70% ethanol. The nails were trimmed and buprenorphine (0.05 mg/ml) was injected intramuscularly. The mice were placed on their side and dorsal skin was pinched along the midline. A sterile 4 mm biopsy punch was then used to create clean-cut, symmetrical, full-thickness excisional wounds on either side of the dorsal midline. L-microgel assemblies (L-MAP), D-microgel assemblies (D-MAP), or L/D-microgel assemblies (L/D-MAP (1:1 mixture)) was injected into the wound.

Wounds were imaged daily to follow closure of the wounds. Each wound site was imaged using high-resolution camera (Nikon Coolpix). Closure fraction was determined by comparing the pixel area of the wound to the pixel area within the 10 mm center hole of the red rubber splint. Closure fractions were normalized to Day 0 for each mouse/scaffold sample. Investigators were blinded to treatment group identity during analysis. After wounds healed, mice were sacrificed on the indicated day after wounding, and tissue collected with a ˜5 mm margin around healed wound. The samples were immediately submerged in Tissue-Tek Optimal Cutting Temperature (OCT) fluid and frozen into a solid block with liquid nitrogen. The blocks were then cryo-sectioned by and kept frozen until use. The sections were then fixed with 4% paraformaldehyde in 1×PBS for 30 minutes at room temperature, washed with 1×PBS, and kept at 4° C. until stained. For antibody production analysis, blood harvested via cardiac puncture to obtain serum for ELISA.

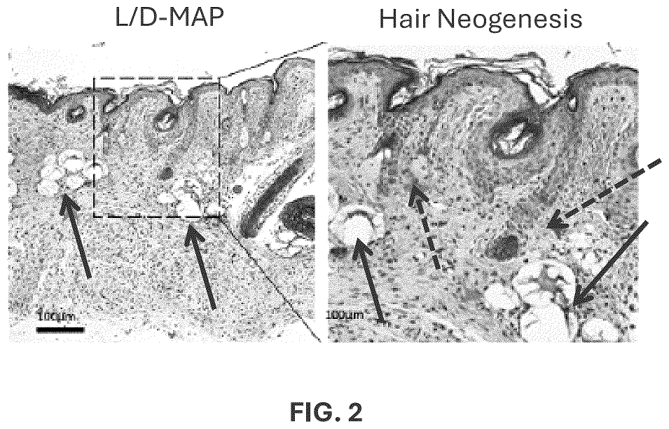

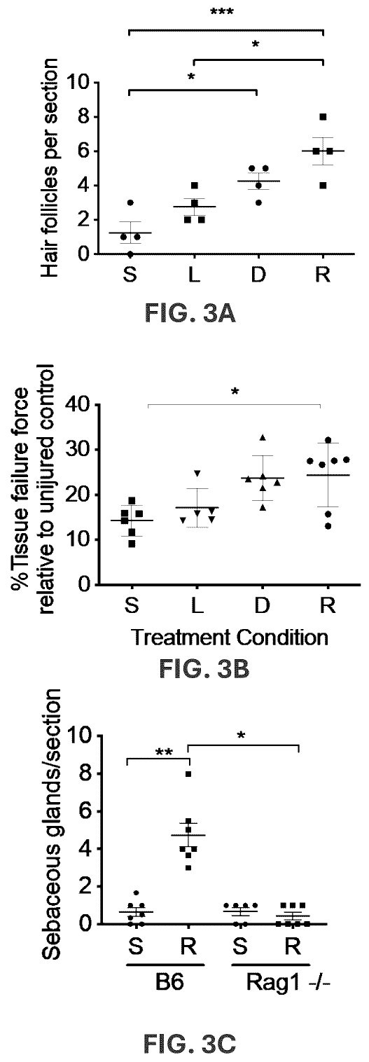

Experimentally induced wounds were treated with L-microgels (L-MAP), D-microgels (D-MAP), or L/D-microgels (L/D-MAP). The inherent porosity of the microgel assembly enhanced cellular infiltration as compared to control animals. L/D-MAP showed an increase in hair neogenesis. (FIG. 2; black arrows show MAP; hatched arrows show neogenic hair follicles). Wound treatment with D-MAP or a 1:1 mixture of L- and D-microgels (R-MAP) led to an increase in hair follicles per section. (FIG. 3A) Mice receiving D-MAP following wound induction showed an increased failure force as compared to mice receiving sham hydrogel or L-MAP or L/D-MAP. The R-MAP induced enhanced skin regeneration, neogenesis of follicles and sebaceous glands, and tensile strength as compared to mice receiving L- or D-MAP. (FIG. 3B) L/D-MAP also showed a statistically significant increase in sebaceous gland regeneration in B6 mice, but not in B6.flag1^{-/-} mice as compared to mice receiving a sham hydrogel. (FIG. 3C)

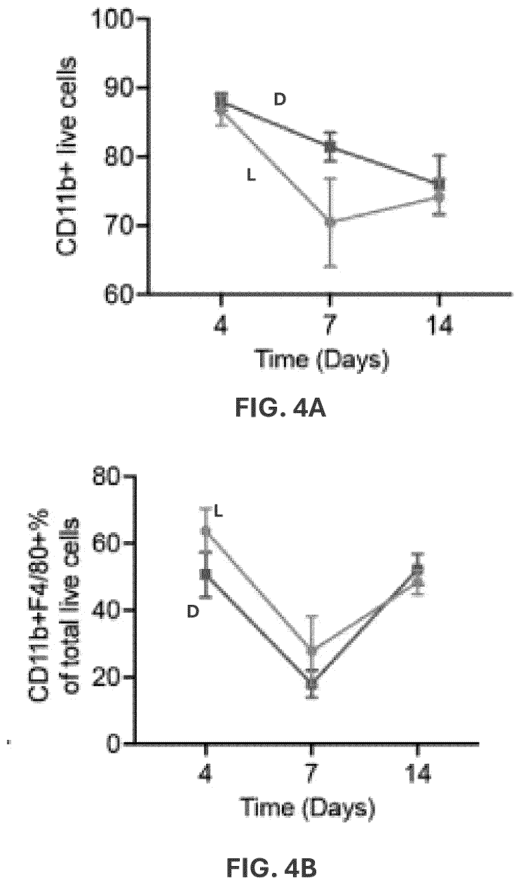

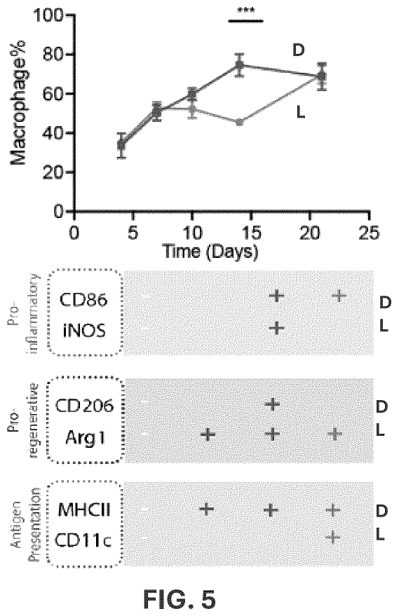

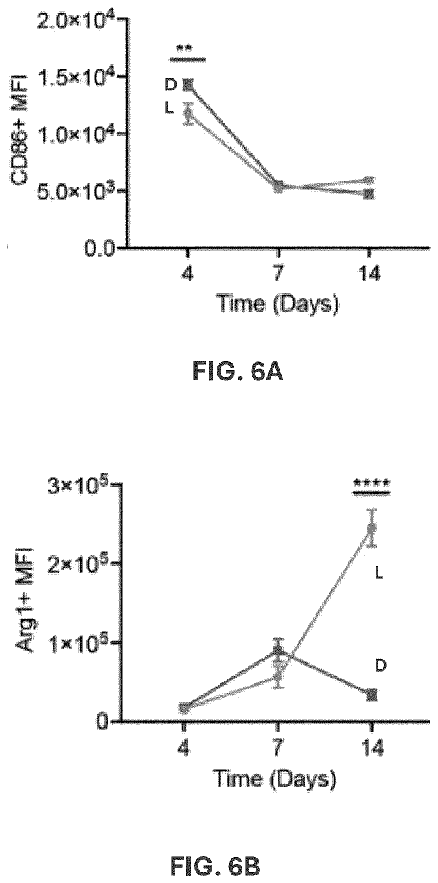

In normal wound healing, CD11b⁺ cells invade quickly during the inflammatory phase but are reduced during proliferation and resolution stages. Treatment of wounds with porous microgel assemblies composed of L-microgels led to an initial enhanced recruitment of CD11b⁺ cells as compared to wound treated with a chemically identical, non-porous hydrogel. Wounds treated with D-microgel assemblies had sustained, elevated recruitment CD11b⁺ cells at 4 and 7 days as compared to wounds treated with L-MAP. (FIG. 4A; **means p<0.01) The percentage of F4/80⁺ cells of live CD11b⁺ cells is shown in FIG. 4B (**means p<0.01). Analysis of subcutaneous implants shows 60% of the CD45⁺ cells are APCs (CD11c⁺/MHCII⁺). (FIG. 5) Expression of CD86 and Arg1 (characteristic of M1 and M2 macrophages) was determined by flow cytometry and compared by mean fluorescence intensity (MFI). (FIGS. 6A and 6B, respectively).

Bone marrow-derived macrophages (BMDM) were prepared from B6 mice and differentiated according to established methods. Briefly, BMDMs were isolated from 8-12 weeks old male C57BL/6 in accordance with institutional and state guidelines and approved by the Duke University's Division of Laboratory Animal Resources (DLAR). Animals were anesthetized, and the bone marrow was extracted from the tibia and femur. Then the bone marrow was broken into cell suspension and the cells are differentiated in culture medium (10% heat inactivated fetal bovine serum, in Iscove's Modified Dulbecco's Medium (IMDM) (Gibco) with Macrophage colony-stimulating factor (M-CSF) (Peprotech). The medium was changed on day 4, day 7, and day 10. After full maturation, the cultured cells were tested for murine macrophage pan marker CD11b and F4/80 by flow cytometry to determine the efficiency.

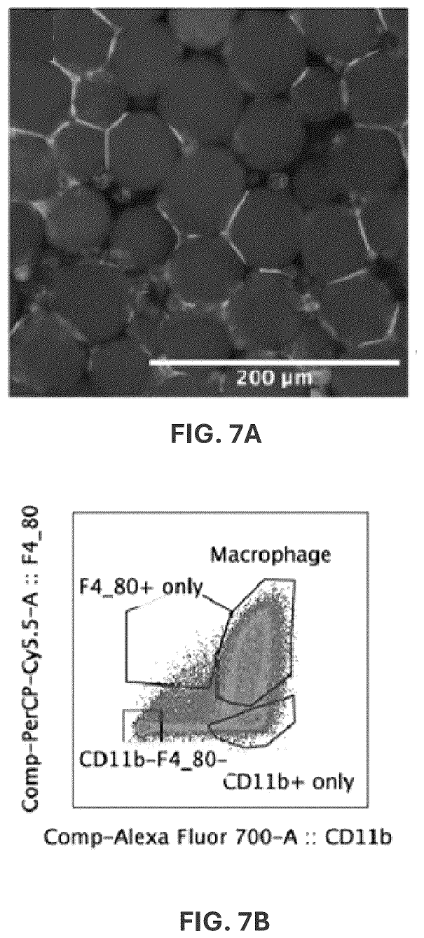

The BMDMs were used in an in vitro, three-dimensional microgel assembly cell culture. Within the microgel assembly, BMDMs exhibited the characteristic morphology (FIG. 7A) and surface proteins (CD86⁺/F4/80⁺) (FIG. 7B).

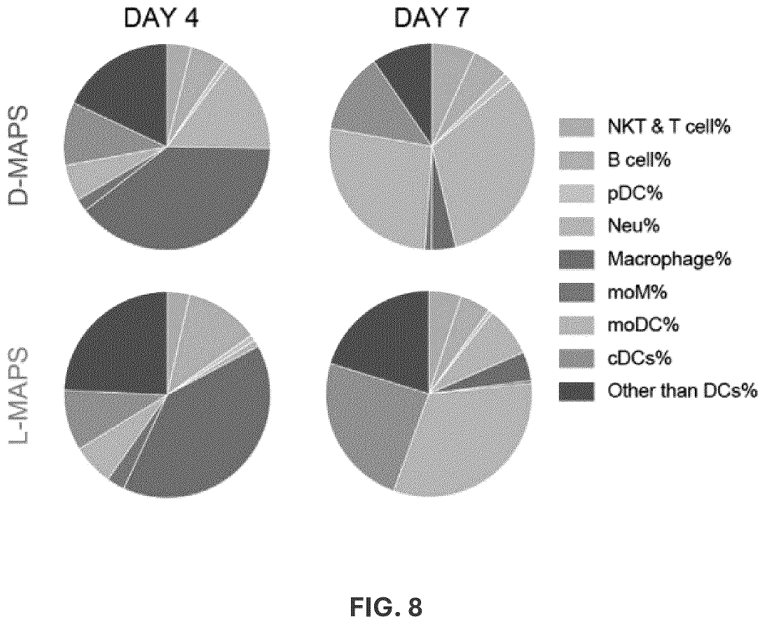

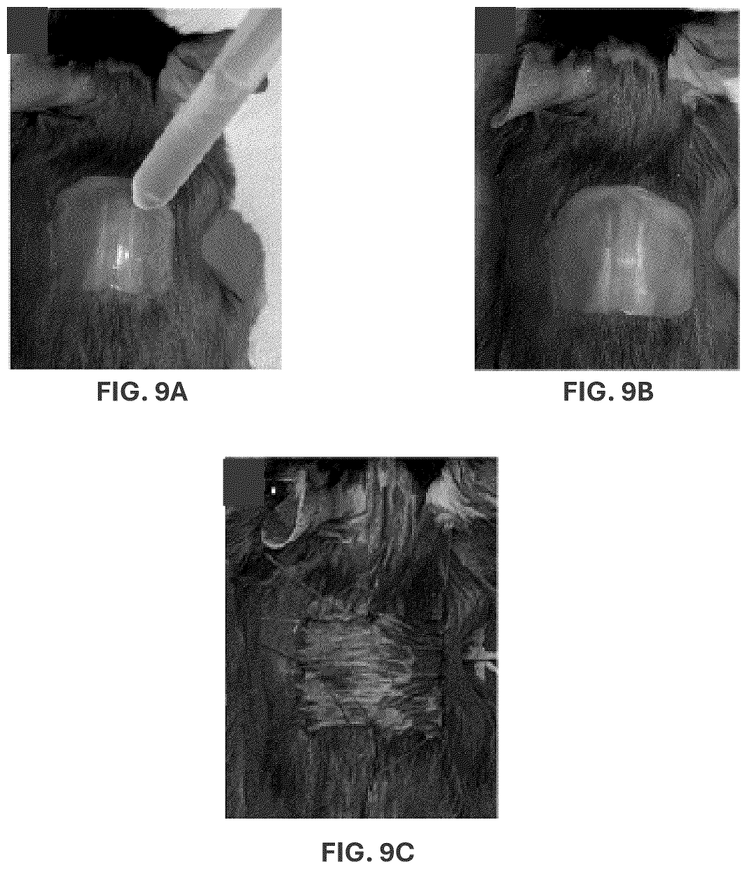

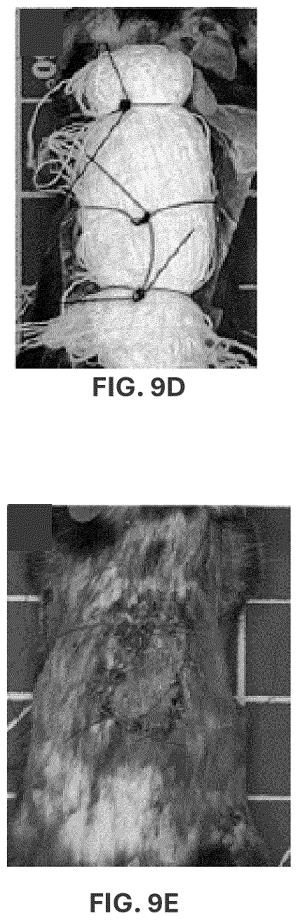

EXAMPLE 2: Site-specific delivery of CTLA4-Ig directs leukocytes towards tolerogenic phenotypes. L- or D-microgel assemblies were prepared as above and fluorescently labeled with AlexaFluor647 (AF647). The L- and D-microgel assemblies were injected subcutaneously, and tissues were analyzed by flow cytometry on Days 4, 7, 14, and 21 following injection. Leukocytes were harvested at Day 7 post injection (POD7). The leukocytes population was assessed and shown in FIG. 8.

Donor PepBoy mice are on a B6 background (H-2^{b}) with all hematopoietic cells expressing CD45.1. Wild-type B6 mice express a different isoform, CD45.2. A soft tissue defect was induced in a wild-type, CD45.2⁺ mouse and donor CD45.1⁺ skin graft was transplanted with or without L/D-MAP treatment as a wound contact layer. Briefly, donor back fur was clipped, prepped with DuraPrep (3M, St. Paul, MN) and allowed to dry for three minutes. One donor animal was sacrificed, and its dorsal back skin prepared to furnish full-thickness skin grafts for four recipients. Recipient skin defects (1.0×1.0 cm) were created in a plane superficial to the panniculus carnosus. Similarly, using sharp dissection under loupe magnification, donor graft tissues were debrided of panniculus carnosus, as reported by others. Grafts were stored in saline-moistened gauze in a petri dish on ice until transplant. Cold ischemia time was approximately 30 minutes per graft (equivalent to in vivo gelation time of microgel assemblies); warm ischemia time was less than 20 minutes per graft. Grafts were affixed to surrounding skin with eight interrupted sutures of 5-0 polypropylene (Covidien, Dublin, Ireland). Bolstering was accomplished with a skin-contact layer of Xeroform (Covidien) followed by sterile dry gauze, Tegaderm (3M) and bolstered with 2-0 silk (Covidien) in the far-near pulley technique. Bolsters were kept in place until postoperative day 4 (POD4). Subsequently, animals were weighed and photographed daily with a 12-megapixel iphone XS (Apple, Cupertino, CA) mounted on a Selfie Ring Light Tripod (Ubeesize, City of Industry, CA). Graft survival was adjudged via photographic review by a board-certified plastic and reconstructive surgeon blinded to treatment conditions.

Animals were sacrificed by carbon dioxide asphyxiation and/or cervical dislocation according to IACUC protocols. Three minutes prior to sacrifice, forelimb footpads were intradermally injected with 10 μL of 1% isosulfan blue dye (Mylan, Canonsburg PA) to facilitate visual identification of axillary lymph nodes. Spleens, axillary and inguinal lymph nodes were collected in sterile PBS with 2% fetal calf serum and mechanically passed through 70-μm cell strainers (VWR, Radnor PA); bone marrow (BM) was flushed from bilateral femurs and tibias after epihyscal osteotomy. Red blood cells in splenic and BM tissues were lysed with ACK buffer (ThermoFisher). Cells were enumerated on a Countess II FL automated cell counter (ThermoFisher) according to manufacturer specifications. The inferior half of skin grafts (and a 3 mm peripheral edge of recipient skin) was minced in RPMI 1640 (ThermoFisher) containing DNAase I (200 U/mL) and collagenase IV (125 U/mL) (Worthington) and shaken at 170 rpm for 30 minutes at 37° C. Digestion was quenched with 10% fetal calf serum in PBS, tissue was passed through 70 μm cell strainers, washed and counted as described above.

Initial CD45.1⁺APC analyses focused on costimulation markers (CD80, CD86), and MHC-II. Cells were stained in round-bottom 96-welled plates (Corning, Corning, NY) and analyzed on a Cytek NL-3000 Flow Cytometer (Cytek Biosciences, Fremont, CA). Data was acquired using SpectroFlo (Cytek) and analyzed with SpectroFlo and FlowJo 8 (BD, Franklin Lakes, NJ). Singlet leukocytes were identified by forward and side scatter, and live cells were identified via ZombieNIR (BioLegend, San Diego CA) according to manufacturer specifications. APC panel markers included CD45.1, CD45.2, CD11b, CDllc, Ly6G and characterized by median fluorescence intensity of CD80, CD86 and MHC-II expression. Lymphocytes were characterized by surface CD3, CD4, CD8, CD25, CD44, CD62L and Foxp3 intracellular expression, using fluorescence minus one (FMO) controls. Intracellular staining was performed with Truc-Nuclear™ Transcription Factor Buffer (BioLegend) according to manufacturer specifications. Flow buffer was 1×PBS (Sigma) with 1 mM EDTA (Sigma), 0.2% bovine serum albumin (Sigma), and 0.025% Proclin (Sigma). Using FMO controls, gating on live singlets, excluding Ly6G⁺ granulocytes, we identified a population of CD11b⁺CD11c⁻ (monocytes) or CD11b⁺CD11c⁺ dendritic cells (DCs) and calculated median fluorescence intensities (MFI), normalized to organ-matched samples from untransplanted, nonoperative controls sacrificed and flow-analyzed on the same day.

Tissue Preparation for Histology was bedded in Optimal Cutting Temperature (OCT) compound (ThermoScientific) and snap-frozen in isopentane cooled to −78° C. in a dry ice/acetone slurry. Additional tissue was harvested and fixed for 4 hours in 4% paraformaldehyde/PBS, embedded in paraffin, sectioned and stained with hematoxylin and cosin in the standard fashion.

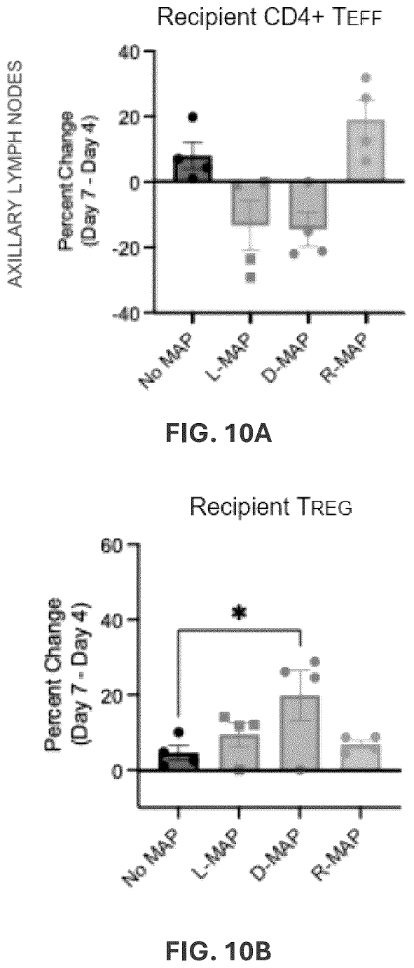

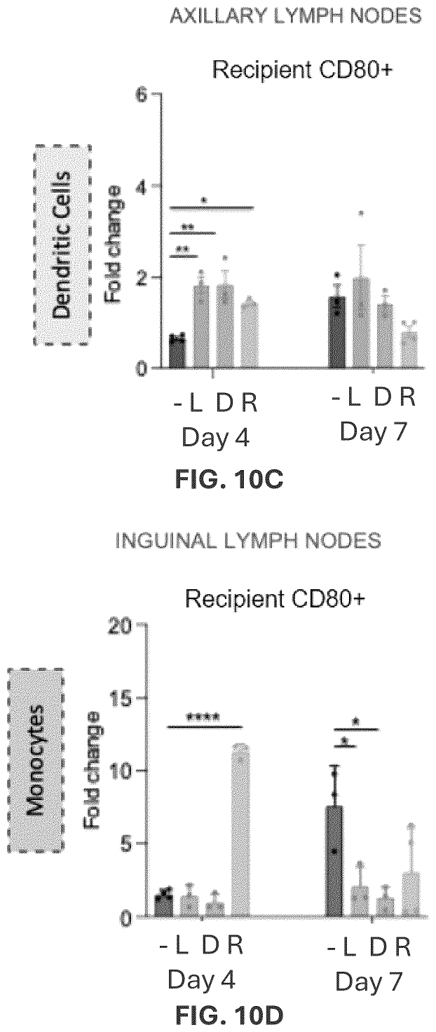

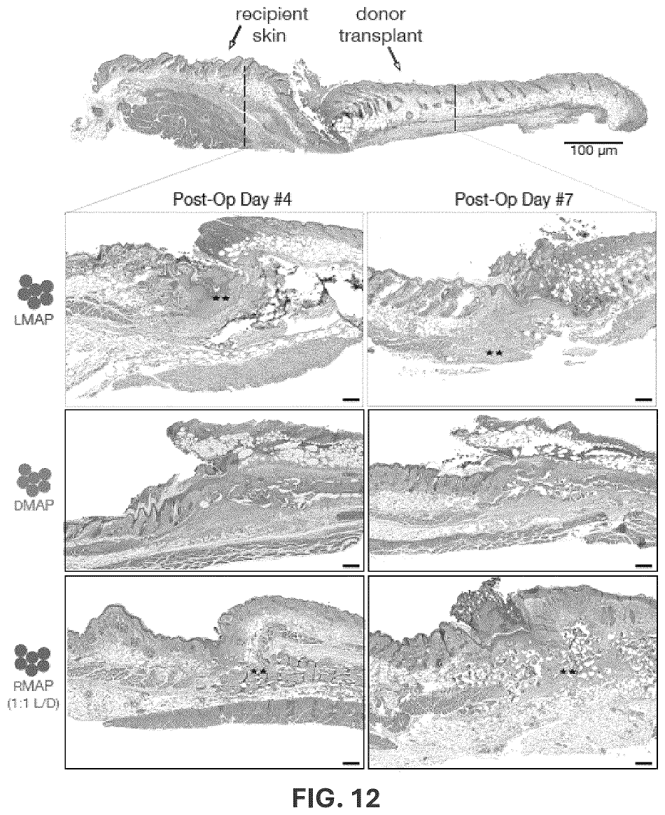

Skin graft with L/D-MAP is shown in FIGS. 12A-12E. Donor- or recipient-derived APCs and memory, naïve, and regulatory T cells in skin, lymph node, spleen, and bone marrow were distinguished by flow cytometry. MAP-treated skin grafts increased immune communication between host and donor. In MHC-matched transplant, L-MAP-augmented skin grafts were non-inferior to no-MAP controls by photographic analysis. At POD7, L/D-MAP-treated recipients showed enhanced presence of CD25⁺/Foxp3⁺ regulatory T cells (T_{Regs}) in axillary draining lymph nodes (21% vs 10% of CD4⁺ T cells, p=0.004) and spleen (8% vs. 15%, p=0.002). Additionally, L/D-MAP transplant recipients show increased draining lymph node-trafficking of CD44⁺/CD62L-effector T cells (T_{eff}) (38% vs 19% of CD4⁺ T cells, p=0.001). (FIG. 10A-10B). Donor and recipient APCs in the bone marrow of the animals treated with L-MAP show elevated MHCII expression in recipient CD80⁺ dendritic cells (FIG. 10C) and donor CD86⁺ monocytes (FIG. 10D) on days 4 and 7 post-transplant.

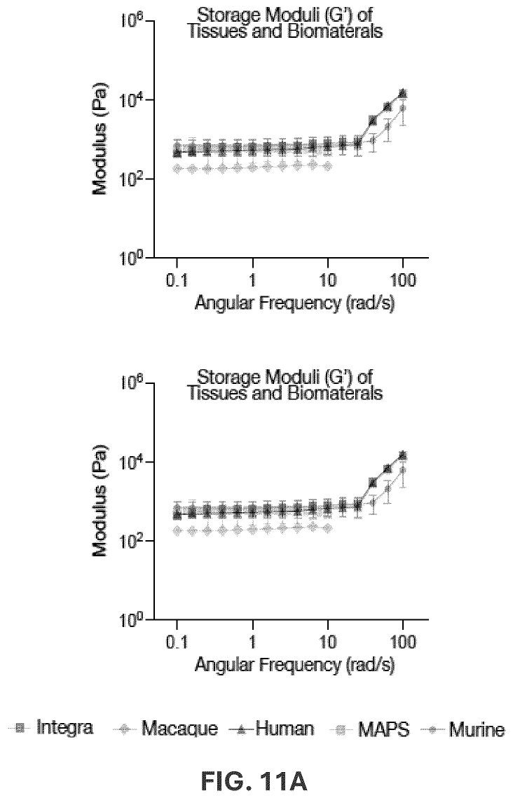

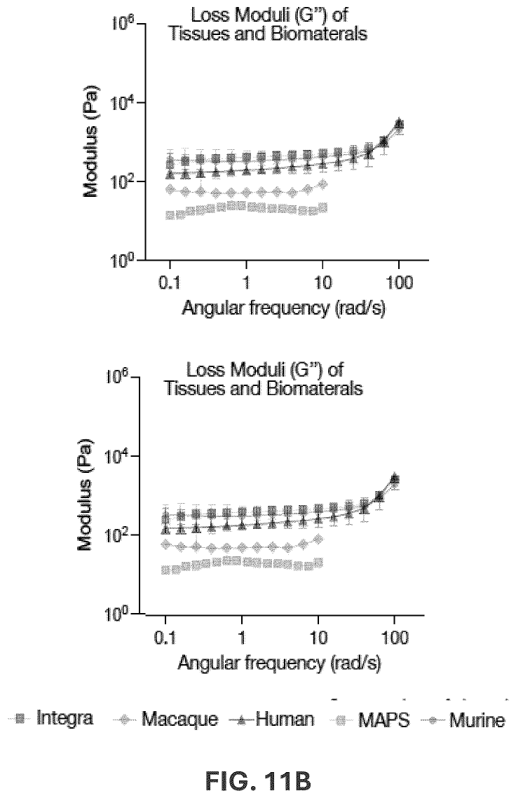

Example 3: Rheology of MAP and animal tissue. Microgel assemblies may be configured to anneal into a viscoelastic solid at euthermic mammalian temperatures (e.g., 37° C.) with a G′ (storage modulus) greater than its G″ (loss modulus). The biomechanical similarity of microgel assemblies with various mammalian skin tissue was measured. Briefly, storage and loss moduli of biomaterials were determined by rotational rheometry (Anton-Parr, Graz, Austria). Frequency sweeps were carried out at a shear range from 10⁻¹ rad·s⁻¹ to 10² rad·s⁻¹ with strain amplitude of 1%. Microparticle size is calculated as the mean of a normalized population sourced from nine images of HMPs, photographed by 4×Ti Eclipse microscope (Nikon, Tokyo, Japan), and analyzed using MATLAB (MathWorks, Natick MA). Human tissues were obtained from clinical collaborators with oversight and sponsorship of the Duke University Institutional Review Board. Tissues sourced from rhesus macaques was obtained from basic science collaborators at Duke University and conformed to all local and federal regulations regarding care and use of non-human primates. The annealed microgel assemblies possess similar stress/strain curves as that of human, mouse, and macaque tissue. (FIG. 11A-11B)

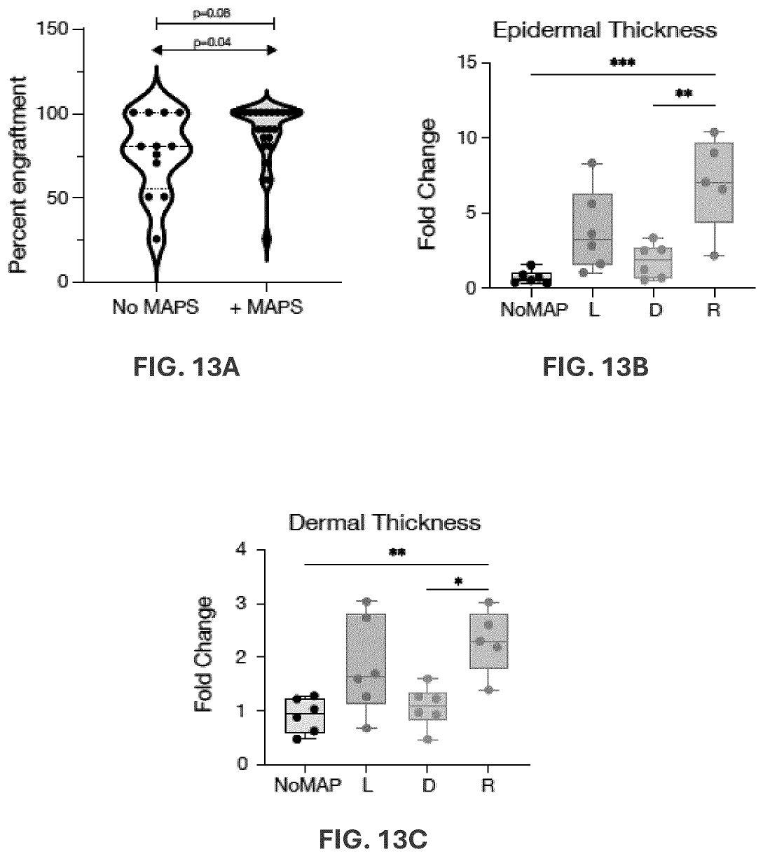

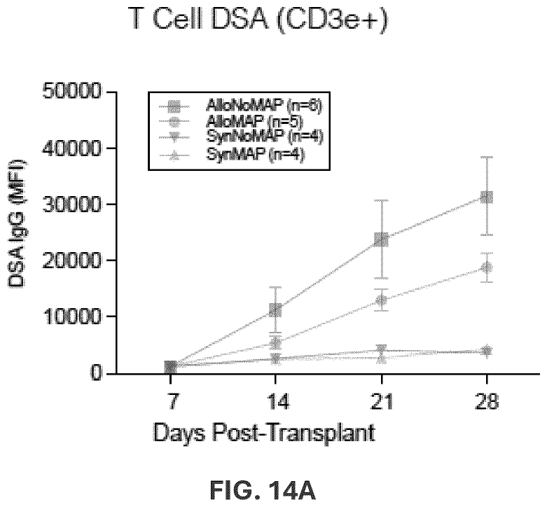

Example 4: MAPs support cumorphic skin regeneration. MHC-matched CD45.1⁺ donor tissue was transplanted to CD45.2⁺ recipient mice as described above. L-microgel assemblies, D-microgel assemblies, or L/D-microgel assemblies were injected to the wound prior to skin graft as a wound contact layer. Wounds were created with sharp microdissection to preserve intact fascia overlying the latissimus dorsi. Panniculus carnosus was removed from donor skin under loupe magnification. Standardized photography was used to analyze skin engraftment. Animals were imaged on day of surgery, upon bolster removal at postoperative day 4 (POD4), then daily until sacrifice. Per animal, series were blinded of metadata (i.e. untreated or treated with MAP) then standardized images were shown to three board-certified surgeon observers to grade the rate (0 to 100%) of skin graft acceptance. A two-sided Mann-Whitney U test was performed (p=0.0835, outward-facing arrows), indicating that skin engraftment was not significantly different between groups. However, ‘no significant difference’ is not the same as equivalence. To address equivalence, the two one-sided tests (TOST) method was employed, conferring a p-value of 0.0426 (inward-facing arrows), indicating the treatment groups are not equivalent. Allograft biopsies and adjacent tissues were analyzed with conventional histopathology and photography at days 4 and 7 post allograft to quantify engraftment. Dermal and epidermal regeneration was observed in all MAP-treated animals. (FIG. 12). Mice treated with microgel assemblies showed a higher percent engraftment as compared to mice that did not receive a microgel assembly. (FIG. 13A) Mice treated with L/D-MAP (R-MAP) had the greatest increase in epidermal and dermal thicknesses. (FIGS. 13B and 13C, respectively)

MAPS-mediated skin allograft attenuates the magnitude, rate, and variability of humoral immunity in MHC-mismatched allograft model. The MHC-mismatch skin transplantation model was similar to above, except that the donor tissue originated in a Balb/c mouse having an H-2^{d} MHC. Recipient B6 mice have H-2^{b} MHC. MHC-mismatched grafts were rapidly rejected in transplant recipients without MAP, as shown by contracture of graft size over the first week following the allograft. Without MAP, recipients demonstrate rapid development of high titers for donor-specific antibodies, culminating in allograft loss.

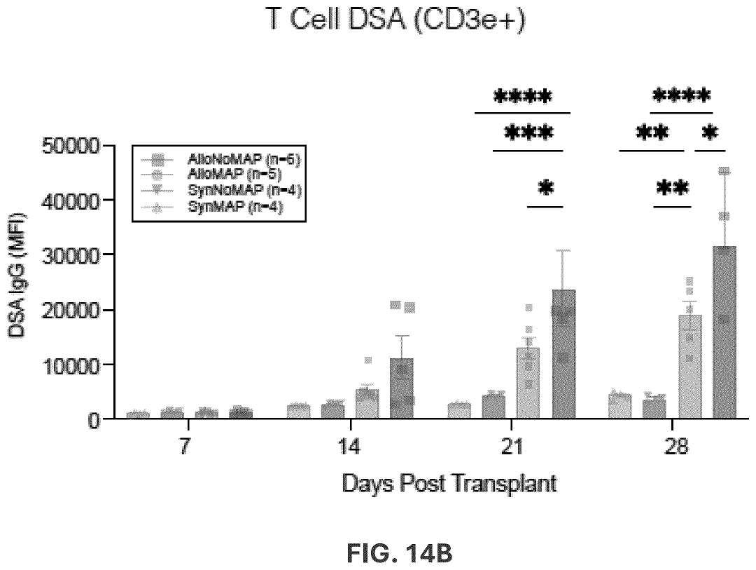

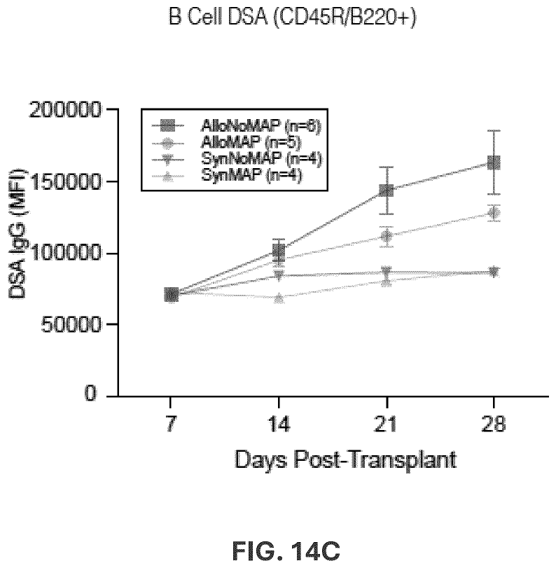

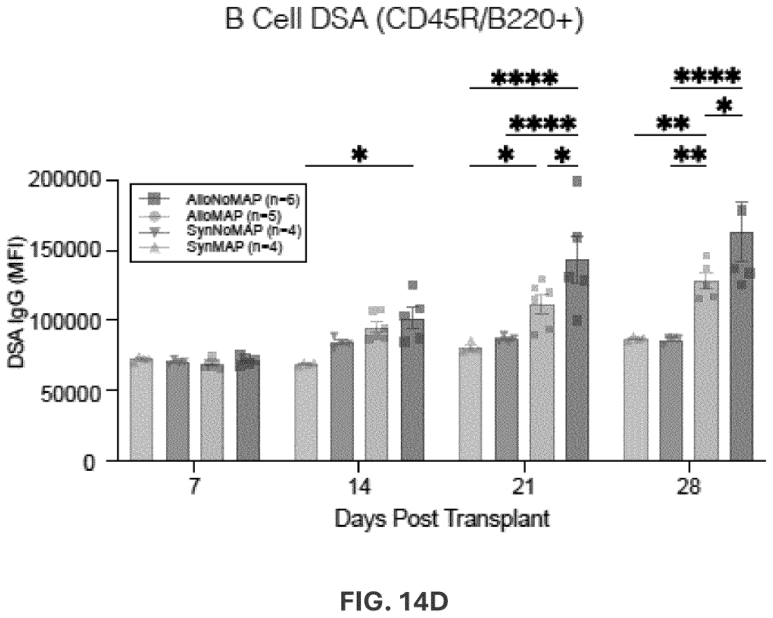

In L/D-MAP-assisted matched skin transplants, lower levels of donor-specific antibodies (DSA) were observed across 4 weeks for all syngeneic mice regardless of MAP condition. Anti-T cell and anti-B cell DSA rose over time in all allogeneic groups regardless of microgel assembly treatment. L/D-MAP attenuated alloreactive DSA in recipient serum, especially three- and four-weeks post-transplant, suggestive of the development of a memory-like response. L/D-MAP reduced the variability of DSA presence in comparison to the AlloNoMAP group. L/D-MAP also slowed the rate of DSA generation in allogeneic transplants. At Day 28 post allograft, the presence of IgG DSA across allogeneic (AlloMAP and AlloNoMAP) and syngeneic skin transplants (SynMAP and SynNoMAP) was assessed via flow crossmatch on donor T and B cells. Mean fluorescence intensity on CD3e⁺Ig⁺ T cells or CD45R⁺ B220⁺Ig⁺ B cells was determined on post-operative days 7, 14, 21, and 28. Anti-T cell and anti-B cell DSA did not increase over four weeks in syngeneic allograft. Anti-T cell and anti-B cell DSA increased dramatically in mice receiving the allogeneic allograft without microgel assembly treatment. The presence of L/D-microgel assemblies prior to skin graft significantly reduced anti-T cell and anti-B cell DSA. (FIGS. 14A-14D)

One skilled in the art will readily appreciate that the present disclosure is well adapted to carry out the objects and obtain the ends and advantages mentioned, as well as those inherent therein. The present disclosure described herein are presently representative of disclosed embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A method of engrafting tissue in a subject, the method comprising:
providing tissue to a location on or in the subject; and
providing a microgel assembly to the location,
wherein the microgel assembly comprises a plurality of microgels
wherein each microgel comprises microgel particles, peptides, and one or more immune modifying agent,
wherein the microgel particles comprise at least one of polyethylene glycol polymers and hyaluronic acid polymers,
wherein each microgel independently comprises only D-amino acids (D-microgels) or only L-amino acids (L-microgels),
wherein the microgel assembly comprises only D-microgel (D-microgel assembly), only L-microgel (L-microgel assembly) or a mixture of D-microgel and L-microgel (L/D-microgel assembly).

2. The method according to claim 1, wherein the one or more immune modifying agent modulates one or more of PD-1, a PD-L1, CTLA4, 4-1BB, CD40L, calcineurin, inosine-5′-monophosphate dehydrogenase, mTOR, JAK, and combinations thereof.

3. The method according to claim 2, wherein the immune modifying agent is a CTLA4 inhibitor.

4. The method according to claim 3, wherein the CTLA4 inhibitor is belatacept.

5. The method according to claim 1, wherein the microgel particles are polyethylene glycol polymers.

6. The method according to claim 1, wherein the microgel assembly is L/D-microgel assembly.

7. The method according to claim 6, wherein the L/D-microgel assembly comprises a 1:1 mixture of D-microgels and L-microgels.

8. The method according to claim 1, wherein the peptides are selected from the group consisting of Ac-FKGGERCG-NH₂ (SEQ ID NO: 1), Ac-NQEQVSPLGGERCG-NH₂ (SEQ ID NO: 2), Ac-RGDSPGERCG-NH₂ (SEQ ID NO: 3), and Ac-GCRDGPQGIWGQDRCG-NH₂ (SEQ ID NO: 4).

9. The method according to claim 1, wherein the microgel assembly further comprises thrombin, Factor XIII, or a combination thereof.

10. The method according to claim 1, wherein the tissue is selected from the group consisting of skin, kidney, bone, liver, spleen, heart, lung, cornea, muscle, ligament, tendon, fascia, nerve, lymph, blood vessels, and combinations thereof.

11. The method according to claim 1, wherein the tissue is allogeneic to the subject.

12. The method according to claim 1, wherein the tissue is syngeneic to the subject.

13. The method according to claim 1, wherein the microgel assembly is provided to the subject before the tissue is provided to the subject.

14. The method according to claim 1, wherein the microgel assembly is applied to the tissue prior to providing the engrafted tissue to the subject.

15. The method according to claim 1, further comprising administration of an immunosuppressive agent.

16. The method according to claim 1, wherein the microgel assembly comprises:
Belatacept or CTLA4-Ig;
polyethylene glycol polymers;
a 1:1 mixture of D-beads and L-beads;
thrombin; and
Factor XIIII,
and wherein the engrafted tissue is skin.

17. A method for reducing an alloreactive immune response to engrafted donor tissue in a subject, the method comprising:
providing the donor tissue to a location on or in the subject; and
providing a microgel assembly to the location,
wherein the microgel assembly comprises a plurality of beads
wherein each bead comprises microgel particles, peptides, and one or more immune modifying agent,
wherein the microgel particles comprise at least one of polyethylene glycol polymers and hyaluronic acid polymers,
wherein each bead independently comprises only D-amino acids (D-microgels) or only L-amino acids (L-microgels),
wherein the microgel assembly comprises only D-microgels (D-microgel assembly), only L-microgels (L-microgel assembly) or a mixture of D-microgels and L-microgels (L/D-microgel assembly),
wherein a host immune response to the engrafted tissue is reduced.

18. The method according to claim 17, wherein microgel assembly is L/D-microgel assembly.

19. The method according to claim 17, wherein the number of alloreactive CD45R⁺ B cells and/or alloreactive CD3⁺ T cells is reduced.

20. The method according to claim 17, wherein the microgel assembly induces tolerance.
